(19) United States
(12) Patent Application Publication
(10) Pub. No.: US 2013/0066422 A1
(43) Pub. Date: Mar. 14, 2013

(54) HAPTIC DEVICE FOR SULCUS IMPLANT

(71) Applicants: Rüdiger DWORSCHAK, Deidesheim (DE); László KONTUR, Munich (DE)

(72) Inventors: Rüdiger DWORSCHAK, Deidesheim (DE); László KONTUR, Munich (DE)

(73) Assignee: QMP HOLDING GMBH, Mannheim (DE)

(21) Appl. No.: 13/609,339

(22) Filed: Sep. 11, 2012

(30) Foreign Application Priority Data
Sep. 14, 2011 (FR) 11 58196

(51) Int. Cl. A61F 2/16

(52) U.S. Cl. 623/6.51

(57) ABSTRACT

An intraocular lens for implantation in the ciliary sulcus of the eye has an optical part, on the periphery of which are arranged at least two haptic struts in the form of closed loops arranged symmetrically on each side of the optical part. Each loop is formed by at least three segments interconnected by flexion points, an arched upper segment forming the part for contact with the sulcus of the eye, and two lower segments providing the join between the upper segment and the optical part of the lens, to which they are also connected by flexion points. The length of the upper segment is less than the sum of the lengths of the lower segments and of their spacing (D) in the area of their connection to the optical part. The spacing is such that the lower segments converge on each other in the direction of the optical part.

The invention relates to lenses implantable in the eye and generally called intraocular lenses (IOL). It relates more particularly to lenses for implantation in the ciliary sulcus, between the iris and the capsular bag, both when the latter, after ablation of the crystalline lens, is unable to receive a corrective lens, and also when said capsular bag already contains an intraocular lens, the sulcus lens then being intended to correct the residual or resulting deficiencies after the implantation of a main lens in the capsular bag, or else when it is necessary to add new features to the optical system of the eye, such as:
correction of corneal astigmatism,
modification of near sight by means of multifocal lenses,
fitting of a filter for blue light in cases of sensitivity to light.

The disadvantage of the known sulcus lenses is that they have a natural tendency to turn and to move off centre as a result of the very irregular anatomical structure of the ciliary sulcus and the instability of the movements of the latter.

The invention aims to overcome this disadvantage and to make available intraocular sulcus lenses of which the haptics ensure a perfect hold of the optical part, regardless of the anatomical structure of the sulcus of the eye of the patient in whom they are implanted, and which do not pose the risk of rotation and off-centring of the known sulcus lenses.

To this end, the invention lies in an intraocular lens for implantation in the ciliary sulcus of the eye, having an optical part, on the periphery of which are arranged at least two haptic struts in the form of closed loops arranged symmetrically on each side of the optical part, each loop being formed by at least three segments, namely an arched upper segment and two lower segments that provide the join between the upper segment and the optical part, said lower segments converging in the direction of the optical part, characterized in that the segments of each loop are interconnected by flexion points, in that the two lower segments are connected to the optical part of the lens by flexion points, and in that the length of the upper segment is less than the sum of the lengths of the lower segments and of their spacing in the area of their connection to the optical part.

Thus, under the compression forces exerted by the structures of the eye in the direction of the optical part, the haptics cannot deform beyond the point of maximum extension in which the two upper segments are situated in alignment with each other. This provides great flexibility of the upper part of the haptics in contact with the ciliary sulcus while at the same time limiting the deformation to a minimum target diameter, preferably of between 10.5 mm and 12.5 mm. The symmetry of the haptic loops, combined with this limitation of their deformability, eliminates the risk of undesirable rotation of the lens under the effect of the forces exerted thereon (contraction of the internal structures of the eye, movements of the eye, rubbing exerted on the eye).

The invention is also distinguished by the following features:
the haptic loops are joined to the periphery of the optical part by stalks;
the upper segment is formed by at least two portions joined to each other by a flexion point;
in the state of maximum compression of the haptic loops, the overall diameter of the lens is between 10.5 mm and 12.5 mm;
the flexion points between the segments of the haptic loops and the flexion points between said loops and the optical part are produced by thinning the cross section of said segments;
the lens has two groups of two haptic loops distributed symmetrically on the periphery of the optical part;
the stalks have projections or hollows that perform a positioning function;
the edge of the rear face of the optical part has spacing projections or stubs.

The invention will be better understood from the following description of a non-limiting embodiment and by reference to the attached drawings, in which:

FIG. 1 is a skeleton diagram illustrating the particular geometry and kinematics of the haptics according to the invention, for an embodiment in which a haptic loop is formed by four segments that are joined to one another by flexion points, namely two upper segments and two lower segments;

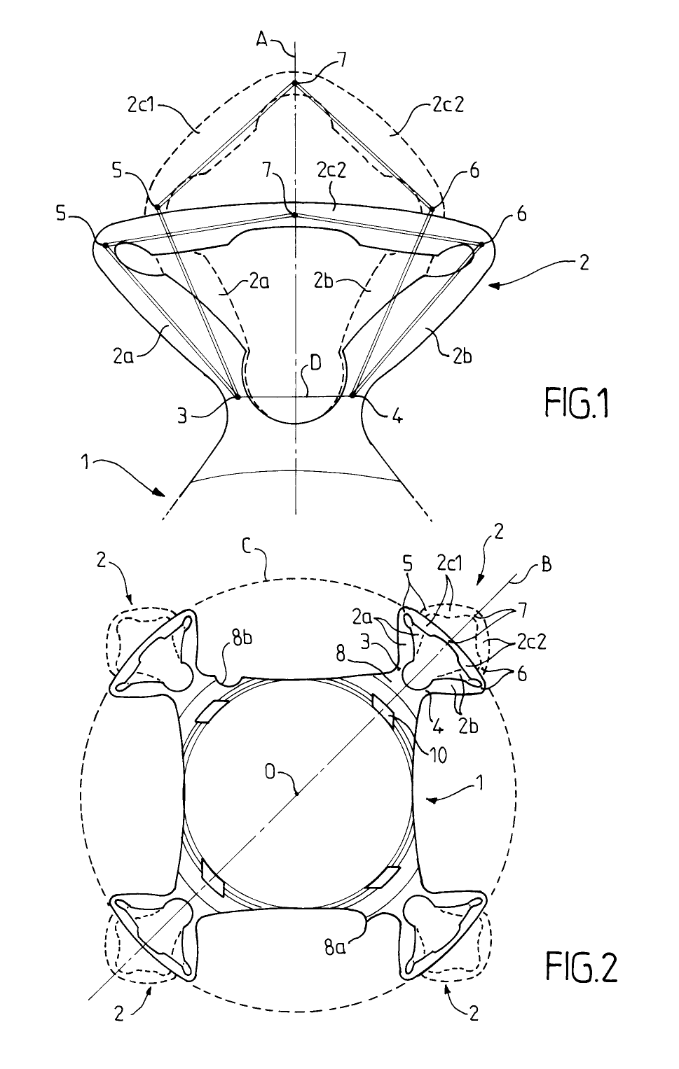

FIG. 2 is a plan view of the posterior face of a sulcus implant according to the invention, in an embodiment in which a haptic loop is formed by four segments that are joined to one another by flexion points, namely two upper segments and two lower segments;

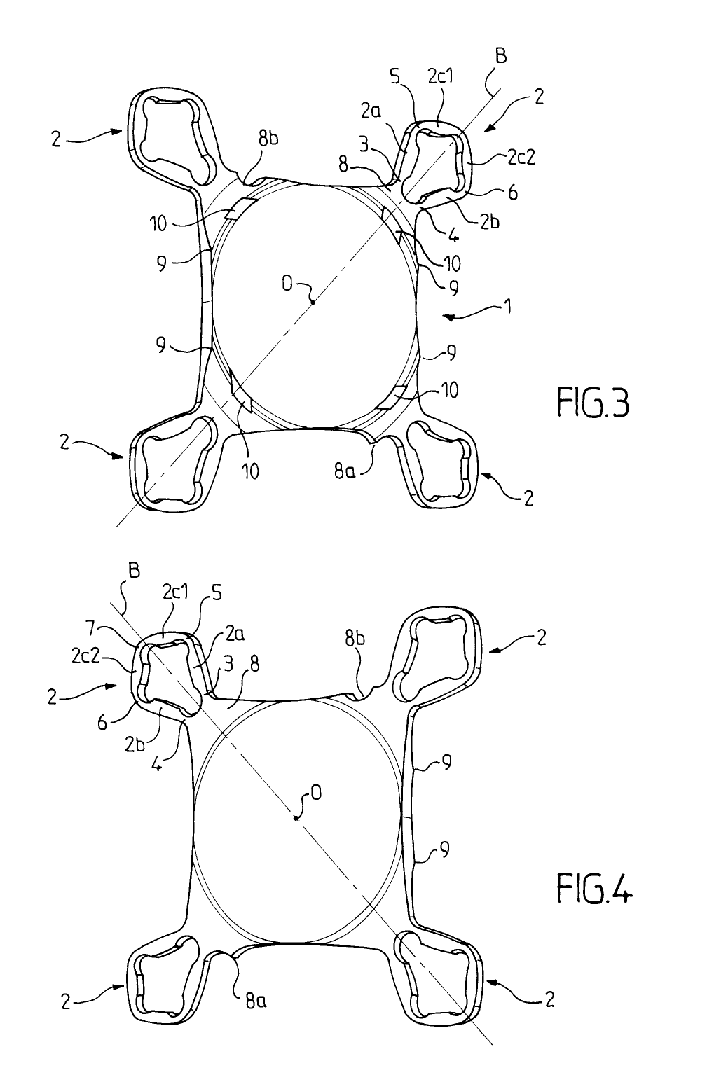

FIG. 3 is a perspective view of the posterior face of the sulcus implant shown in FIG. 2;

FIG. 4 is a perspective view of the anterior face of the sulcus implant shown in FIG. 2;

FIG. 5 is a sectional view in the median vertical plane of the sulcus implant shown in FIG. 2;

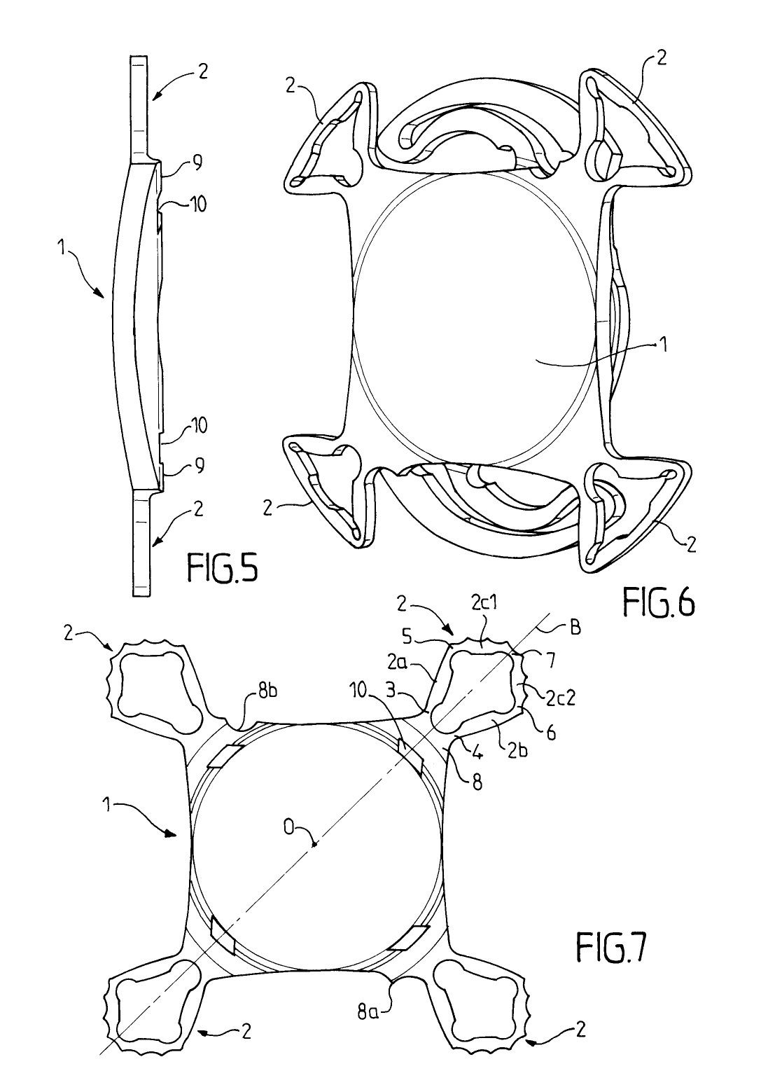

FIG. 6 is a perspective view of the combination of a sulcus implant shown in FIG. 2 with a capsular bag implant;

FIG. 7 is a view similar to FIG. 2, showing a particular embodiment in which the upper part of the haptic loops of the sulcus implant has a crenulated contour;

FIG. 8 is a skeleton diagram illustrating the particular geometry and kinematics of the haptics according to the invention, for an embodiment in which a haptic loop is formed by three segments that are joined to one another by flexion points, namely an arched upper segment and two lower segments;

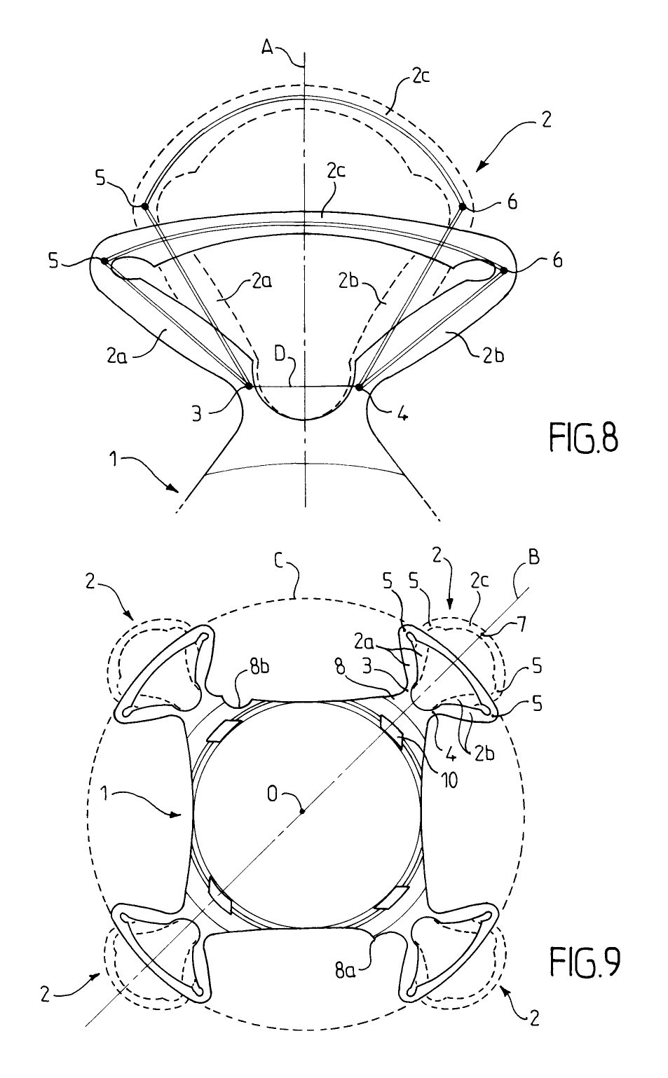

FIG. 9 is a plan view of the posterior face of a sulcus implant according to the invention as shown in FIG. 8.

In the various figures, elements that are identical or have a similar function are indicated by the same references.

In the figures, reference number 1 designates the optical part, and reference number 2 designates the haptic part.

FIG. 1 provides a schematic illustration of a haptic loop according to the invention, at rest (broken lines) and in the state of maximum deformation (solid lines), with the straight line portions illustrating these different positions. It will be seen there that the loop 2 is formed by two lower segments 2a, 2b which, at one end, are connected by two lower flexion points 3, 4 to the periphery of the optical part 1, partially shown in the diagram, and, at the other end, are connected by two upper flexion points 5, 6 to an upper segment formed by two parts 2c1, 2c2, which are joined to each other by a flexion point 7. The lower flexion points 3, are spaced apart by a distance D, such that they converge in the direction of the optical part and diverge in the direction of the upper segment 2c1, 2c2 with respect to the median vertical axis A passing through the pole of a haptic loop 2 and through the optic axis of the lens. The sum of the lengths of the lower segments 2a, 2b and of the distance D is greater than the length of the upper segment 2c1, 2c2. Under the effect of compression forces acting on the pole of the loop 2, substantially at the flexion point 7, in the direction of the optical part, the pivoting of the lower segments 2a, 2b produces the lowering of the upper flexion points 5, 6 and the flattening of the segment 2c1, 2c2. By design, the haptic loop 2 cannot continue to deform beyond the lower position shown in FIG. 1.

Thus, a haptic loop 2 is obtained that can deform elastically in the direction of the optical part by a limited distance, this deformation being blocked when the maximum spacing of the lower segments is reached.

In practice, the dimensions of the segments forming the haptic loop 2 and the spacing D between the points of connection of the lower segments 2a, 2b to the optical part are chosen such that the overall diameter of the lens, in the state of maximum compression of its haptic loops 2, is between 10.5 mm and 12.5 mm.

FIG. 2 shows a plan view of the posterior face of an embodiment of a sulcus implant in accordance with the skeleton diagram of FIG. 1.

The sulcus implant has four haptic loops 2 regularly distributed about the periphery of the optical part 1. Since the haptic loops 2 are identical, the reference numbers have been placed on only one of them, so as not to complicate the drawing. The haptic loops 2 are shown in solid lines (compressed position) and in broken lines (rest position). Each loop 2 is connected to said optical part by a flat stalk 8, which is of generally triangular shape and has a thickness of about 0.2 mm.

The haptic loops 2 form two diametrically opposite groups in relation to a median transverse axis B passing through the optic axis O. The stalk 8 of one of the two haptic loops of each group has a lateral projection 8a or a hollow 8b performing a positioning function.

The concentric circle C symbolizes the position of maximum compression of the haptic loops 2. In practice, the diameter of the circle C will be between 10.5 mm and 12.5 mm.

The lower segments 2a, 2b preferably have a length of the order of 1.6 mm, and the upper segments 2c1, 2c2 a length of the order of 1.4 mm. The flexion points 3, 4, 5, 6 and 7 are preferably obtained by reducing the cross section of the material from which each haptic loop 2 is made.

Under the effect of the compression forces exerted on the haptic loops 2, the latter deform with a gradual flattening movement of the upper segments 2c1, 2c2 and spacing-apart of the lower segments 2a, 2b about flexion points 3, 4, 5, 6 and 7. The size ratio of the lower segments with respect to the upper segments, their spacing from each other and their convergence in the direction of the optic axis O ensure that the movement of deformation does not go beyond a return point at which the upper segments 2c1, 2c2 are substantially in alignment with each other.

The lenses are preferably made of an acrylic material.

The stalks 8 are flat and thin and have the effect of avoiding a risk of the iris being caught by the haptic loops.

The lens shown in FIGS. 2, 3 and 4 is an additional lens for positioning in the ciliary sulcus in front of a capsular bag lens. In a manner known per se, its optical part 1 is concavo-convex, so as to adapt to the front face of the main lens present in the capsular bag. To ensure that a space permitting circulation of the aqueous humor is maintained between the adjacent faces of the optical parts of the main lens and of the additional lens, the rear edge of the optical part of the sulcus lens has four projections or stubs 9 arranged regularly on the perimeter of said optical part. For this same purpose, recesses 10 are additionally arranged on the periphery of the posterior face of the optical part 1 and substantially centred on the diameters passing through the poles of the haptic loops 2.

FIG. 7 illustrates a variant embodiment of a lens according to FIGS. 2 to 6, in which the upper part of the haptic loops 2 of the sulcus implant has a crenulated contour. This arrangement permits a better engagement of this part of the haptic loops with the internal periphery of the ciliary sulcus and, as such, a better hold of the lens.

FIG. 8 shows a schematic view of a second embodiment of a haptic loop according to the invention, at rest (broken lines) and in the state of maximum deformation (solid lines), with the straight line portions illustrating these different positions. In contrast to the schematic view in FIG. 1, in this illustrative embodiment the upper part of the haptic loop is formed by a single arched segment 2c instead of two segments joined by a flexion point. All the other features of the lens are similar and are designated by the same reference numbers. The upper segment 2c is arched such that, under the effect of the compression forces acting in the direction of the optical part, the pivoting of the lower segments 2a, 2b produces the lowering of the upper flexion points 5, 6 and the flattening of the segment 2c. As in the embodiment described in relation to FIGS. 1 to 7, the haptic loop 2 cannot continue to deform beyond the lower position shown in FIG. 8.

FIG. 9 shows a plan view of the posterior face of a lens in accordance with the schematic view in FIG. 8. As in the preceding embodiment, this is an additional lens for positioning in the ciliary sulcus in front of a capsular bag lens. It can likewise be made with a crenulated contour on the face via which the upper part of its haptic loops 2 makes contact with the internal periphery of the ciliary sulcus.

The invention, described here using the example of an additional sulcus implant, is not limited to this embodiment. It is also applicable to any intraocular lens for implantation in the iridocorneal angle of the anterior chamber. Similarly, in the embodiments shown in the figures, the sulcus lenses have four haptic loops regularly distributed on the circumference of the optical part. A lens according to the invention can alternatively have only two or three haptic loops regularly distributed on the circumference of the optical part.

1. Intraocular lens for implantation in the ciliary sulcus of the eye, having an optical part (1), on the periphery of which are arranged at least two haptic struts (2) in the form of closed loops arranged symmetrically on each side of the optical part, each loop (2) being formed by at least three segments, namely an arched upper segment (2c) and two lower segments (2a, 2b) that provide the join between the upper segment (2c) and the optical part (1), said lower segments converging in the direction of the optical part, characterized in that the segments of each loop (2) are inter-connected by flexion points (5, 6), in that the two lower segments (2a, 2b) are connected to the optical part (1) of the lens by flexion points (3, 4), and in that the length of the upper segment (2c) is less than the sum of the lengths of the lower segments (2a, 2b) and of their spacing (D) in the area of their connection to the optical part.

2. Intraocular lens according to claim 1, characterized in that the upper segment (2c) is formed by at least two portions (2c1, 2c2) connected to each other by a flexion point (7).

3. Intraocular lens according to claim 1, characterized in that, in the state of maximum compression of the haptic loops, its overall diameter is between 10.5 mm and 12.5 mm.

4. Intraocular lens according to claim 1, characterized in that the flexion points (5, 6) between the segments (2a, 2b, 2c) of the haptic loops (2) and the flexion points (3, 4) between said loops and the optical part (1) are produced by thinning the cross section of said segments.

5. Intraocular lens according to claim 1, characterized in that it has two groups of two haptic loops (2) distributed symmetrically on the periphery of the optical part (1).

6. Intraocular lens according to claim 1, characterized in that the haptic loops (2) are joined to the periphery of the optical part (1) by stalks (8).

7. Intraocular lens according to claim 6, characterized in that the stalks have projections (8a) or hollows (8b) that perform a positioning function.

8. Intraocular lens according to claim 1, characterized in that the edge of the rear face of the optical part (1) has spacing projections or stubs (9).

9. Intraocular lens according to claim 1, characterized in that the edge of the rear face of the optical part (1) has recesses (10) that are centred substantially on the diameters passing through the poles of the haptic loops (2).

10. Intraocular lens according to claim 1, characterized in that the upper part of the haptic loops (2) has a crenulated contour.

11. Intraocular lens according to claim 2, characterized in that, in the state of maximum compression of the haptic loops, its overall diameter is between 10.5 mm and 12.5 mm.

12. Intraocular lens according to claim 2, characterized in that the flexion points (5, 6) between the segments (2a, 2b, 2c) of the haptic loops (2) and the flexion points (3, 4) between said loops and the optical part (1) are produced by thinning the cross section of said segments.

13. Intraocular lens according to claim 2, characterized in that it has two groups of two haptic loops (2) distributed symmetrically on the periphery of the optical part (1).

14. Intraocular lens according to claim 2, characterized in that the haptic loops (2) are joined to the periphery of the optical part (1) by stalks (8).
